(19) United States
(12) Patent Application Publication
(10) Pub. No.: US 2020/0207695 A1
(43) Pub. Date: Jul. 2, 2020

(54) PREPARATION OF FUNCTIONALIZED ORGANIC MAGNESIUM SALT AND USE THEREOF IN PREPARATION OF POLYESTER COMPOSITE MATERIAL

(71) Applicant: Northwest Normal University, Gansu (CN)

(72) Inventors: Ziqiang Lei, Lanzhou (CN); Jinnan He, Lanzhou (CN); Yaoxia Yang, Lanzhou (CN); Zhiwang Yang, Lanzhou (CN); Wei Zeng, Lanzhou (CN); Jianping Shi, Lanzhou (CN)

(21) Appl. No.: 16/728,998

(22) Filed: Dec. 27, 2019

(30) Foreign Application Priority Data
Dec. 29, 2018 (CN) 201811637445.5

(51) Int. Cl. C07C 51/41; C08L 67/02; C08G 63/183; C08G 63/86

(52) U.S. Cl. CPC C07C 51/412; C08L 67/02; C08L 2201/02; C08G 63/866; C08G 63/183

(57) ABSTRACT

The present invention discloses a method for preparing a functionalized organic magnesium salt. The method includes completely dissolving organic acid in distilled water to form an organic acid aqueous solution, adding an inorganic magnesium salt into the organic acid aqueous solution, magnetically stirring for 1-5 h at 70-100° C., removing distilled water, and performing vacuum drying to obtain a white powdery solid which is a functionalized organic magnesium salt. An intrinsic flame-retardant PET composite material prepared by using the functionalized organic magnesium salt as a flame retardant not only achieves the compatibility between inorganic particles and a matrix, but also solves the problem of poor flame retardant performance of PET plastics in the existing production process.

### TECHNICAL FIELD

The present invention relates to a method for preparing a functionalized organic magnesium salt, and use thereof as a flame retardant in preparation of a flame-retardant PET composite material, belonging to the technical fields of flame retardant materials and polymer material synthesis.

### BACKGROUND

Most of polyester synthesized early was aliphatic polyester products, which had low molecular weight and melting point and could be dissolved in water easily. In 1941, British J. R. Whenfield and J. T. Dikson obtained polyethylene terephthalate with a higher melting point than aliphatic polyester through synthesis by using terephthalic acid and ethylene glycol as raw materials. PET is a linear thermoplastic material, which has been widely used in many fields such as fiber, engineering plastics and film packaging due to its excellent mechanical and thermal properties and low cost. In particular, dacron produced using PET as the raw material is one of synthetic fibers with the largest yield in China. However, the traditional PET has easy flammability and high combustion speed, and cannot be extinguished easily. Moreover, molten drips generated in the combustion process can also ignite other substances, so that the combustion of materials is aggravated, thereby causing serious fire and casualty accidents. Therefore, the prevention of fire is the current hot topic of social research.

In order to improve the safety of synthetic materials, appropriate flame retardants can be added into synthetic polymer materials, so that the synthetic materials can achieve the purpose of flame retardancy and smoke suppression. At present, the flame retardants used for polyester flame retardancy modification mainly contain halogen and phosphorus. Although these two flame retardants have high flame retardant efficiency, halogen flame retardants can release a large amount of smoke and toxic gases in the combustion process, and also generate carcinogenic substances. This will not only pollute the living environment of human beings, but also cause great harm to health. Green and environment-friendly flame retardants are currently the hotspot in the industry. As people pay more attention to environmental protection and health, halogen-free flame retardants are favored by researchers because of their non-toxicity and cost-effectiveness. Among halogen-free flame retardants, inorganic particles can also reduce heat release on the surface of materials besides good smoke suppression, flame retardancy and thermal stability. As an environment-friendly green flame retardant, the inorganic particles are widely used in rubber, plastic and other fields. However, since the shortcomings (high consumption and poor compatibility with a matrix) of the inorganic particles seriously affect mechanical properties of the matrix, ultrafine inorganic particles have become the focus of our current research. Ultrafine inorganic particles can enhance the contact area with the matrix, and reduce the amount of flame retardants used, thus improving the thermal stability of materials, and meeting the needs of different fields.

### SUMMARY

An objective of the present invention is to provide a method for preparing a functionalized organic magnesium salt.

Another objective of the present invention is to provide a method for preparing a flame-retardant polyester composite material using the functionalized organic magnesium salt as a flame retardant.

I. Preparation of a Functionalized Organic Magnesium Salt

A method for preparing a functionalized organic magnesium salt provided by the present invention includes completely dissolving organic acid in distilled water to form an organic acid aqueous solution, adding an inorganic magnesium salt into the organic acid aqueous solution, magnetically stirring for 1-5 h at 70-100° C., removing distilled water, and performing vacuum drying to obtain a white powdery solid which is a functionalized organic magnesium salt.

The organic acid is at least one of phosphorous acid, succinic acid, p-aminobenzoic acid and pimelic acid; the inorganic magnesium salt is magnesium hydroxide, magnesium carbonate, magnesium borate and magnesium oxide having a particle size of 400-600 nm; and a molar ratio of the organic acid to inorganic particles is (0.5:1)-(3:1).

II. Preparation of a Flame-Retardant PET Composite Material

Terephthalic acid, ethylene glycol, antimony trioxide and the functionalized organic magnesium salt are added into a reaction kettle, and esterification reaction is carried out at 210-240° C. and 0.2-0.35 MPa under nitrogen atmosphere; after the esterification is finished, the system enters a vacuum polycondensation stage: the temperature is first controlled at 260-280° C. for low vacuum polycondensation for 0.5-1.5 h, and then the temperature is raised to 270-280° C. for high vacuum polycondensation; when the stirring power no longer increases, the reaction is finished, a material is discharged and subjected to grain-sized dicing, and the master batch is dried at 120-160° C. for 12-24 h to remove moisture, so that a polyester composite material is obtained.

A molar ratio of the terephthalic acid to the ethylene glycol is (1:1.2)-(1:1.5); and the consumption of the catalyst antimony trioxide is 1/10000-2/10000 of the molar weight of the terephthalic acid.

The consumption of the functionalized organic magnesium salt is 1%-10% of the total mass of the polyester.

III. Characterization of the Functionalized Organic Magnesium Salt and the Flame-Retardant PET Composite Material

Inorganic particle magnesium hydroxide is used as an example below. The following describes the structure obtained after the magnesium hydroxide is subjected to organic functionalization with succinic acid and the preparation and properties of the PET composite material containing the functionalized organic magnesium salt.

FIG. 1 is an infrared absorption spectrum of a functionalized organic magnesium salt. As can be seen from FIG. 1, a strong stretching vibration peak observed at the position of 3564 cm⁻¹ is a stretching vibration absorption characteristic peak of O—H in carboxylic acid, and the position of 1690 cm⁻¹ is a stretching vibration absorption peak of carboxyl groups in carboxylic acid. Meanwhile, an —OH characteristic peak of magnesium hydroxide at the position of 3690 cm⁻¹ disappears, indicating that hydroxyl groups at both ends of magnesium hydroxide have reacted and magnesium hydroxyl groups no longer exist in a newly generated magnesium salt.

FIG. 2 illustrates thermal stability analysis of the functionalized organic magnesium salt. As can be seen from FIG. 2, the thermal decomposition temperature of succinic acid (SA) is 150-250° C. Magnesium hydroxide (MH) absorbs heat at high temperature (300-400° C.) and decomposes into magnesium oxide and water vapor. The thermal decomposition of magnesium succinate (SMH) is divided into three stages: (1) The mass loss is caused by crystal water in a product mainly in the range of 50-150° C. (2) The mass loss is caused by dehydration-condensation reaction of diacid in the organic magnesium salt at 150-500° C. (3) The mass loss is caused by thermal degradation in thermal decomposition of organic matter in SMH at 500-650° C.

FIG. 3 is an XRD curve of the functionalized organic magnesium salt. From FIG. 3, it can be seen that the functionalized organic magnesium salt partially retains crystal characteristic diffraction peaks of magnesium hydroxide, while new characteristic diffraction peaks appear at positions of 11.9°, 23.8°, 25.9°, 30.7° and 31.4°. All above indicate that hydroxyl groups at both ends of magnesium hydroxide have reacted with carboxyl groups of succinic acid, and the synthesized product is a crystal with a complete crystal structure.

FIG. 4 shows infrared absorption spectrum curves of a flame-retardant PET composite material and a pure PET. It can be seen from FIG. 4 that main characteristic peaks are similar, and some characteristic peaks overlap. 1600 cm⁻¹ and 1424 cm⁻¹ correspond to symmetrical stretching vibration absorption peaks of a benzene skeleton. 727 cm⁻¹ corresponds to a deformation vibration absorption peak of H on the benzene ring. A strong absorption band appears at the position of 1768 cm⁻¹, which is a stretching vibration peak of ester carbonyl C═O; and an absorption peak at the position of 1174 cm⁻¹ is a (C—O—C) stretching vibration absorption peak. There is a wide absorption peak at the position of 3464 cm⁻¹ in PET, which is an O—H stretching vibration peak of water, while an O—H stretching vibration peak in polyester (PET-1) is weakened to some extent.

FIG. 5 shows peak heat release rate curves of the flame-retardant PET composite material and the pure PET. FIG. 5 shows that the PET has a peak heat release rate of 646 kW/m². With the increase of the content of the organic magnesium salt, the peak heat release rate of the composite decreases obviously. When 6% of organic magnesium salt is introduced, the peak heat release rate decreases to 403 kW/m², and the peak heat release rate decreases by 37.6%. The lower the peak heat release rate, the less the heat the material transfers to the surface during combustion, which can prevent further thermal decomposition of the material and inhibit the combustion reaction.

FIG. 6 shows total heat release amount curves of the flame-retardant PET composite material and the pure PET. From FIG. 6, it can be seen that the total heat release amount of the polyester composite material after the introduction of the organic magnesium salt is significantly less than that of the pure PET. The total heat release amount of the PET is 86 MJ/m², while the total heat release amounts of polyester (PET-1), polyester (PET-2) and polyester (PET-3) are 71 MJ/m², 69 MJ/m² and 62 MJ/m² respectively. This means that the introduction of the organic magnesium salt delays the burning speed of the material, improves the flame retardant performance of the material and reduces the frequency of fire accidents.

### BRIEF DESCRIPTION OF THE DRAWINGS

FIG. 1 is an infrared absorption spectrum of a functionalized organic magnesium salt prepared according to the present invention;

FIG. 2 illustrates thermal stability analysis of the functionalized organic magnesium salt prepared according to the present invention;

FIG. 3 is an XRD curve of the functionalized organic magnesium salt prepared according to the present invention;

FIG. 4 shows infrared absorption spectrum curves of a flame-retardant polyester composite material and a pure polyester prepared according to the present invention;

FIG. 5 shows peak heat release rate curves of the flame-retardant polyester composite material and the pure polyester prepared according to the present invention; and

FIG. 6 shows total heat release amount curves of the flame-retardant polyester composite material and the pure polyester prepared according to the present invention.

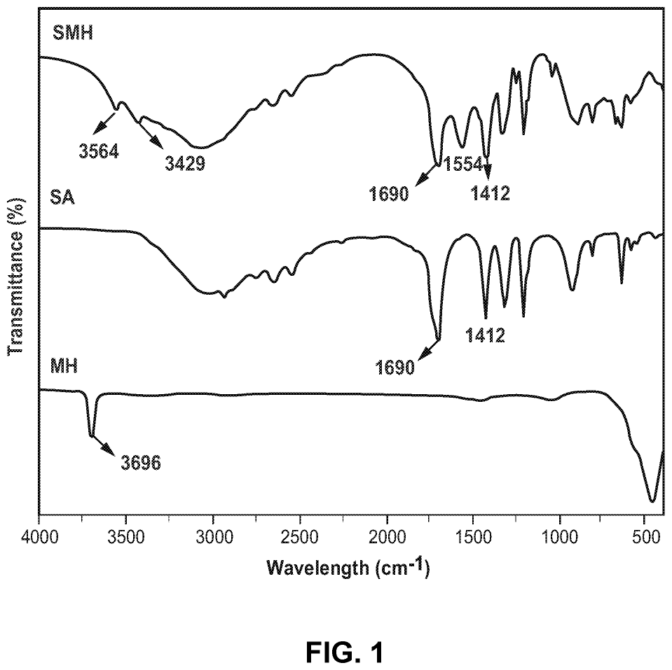

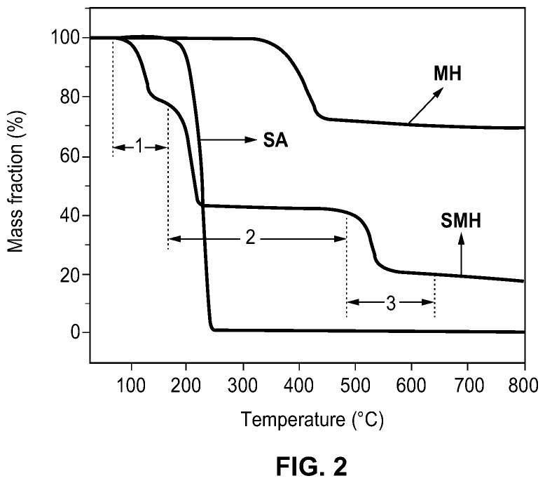

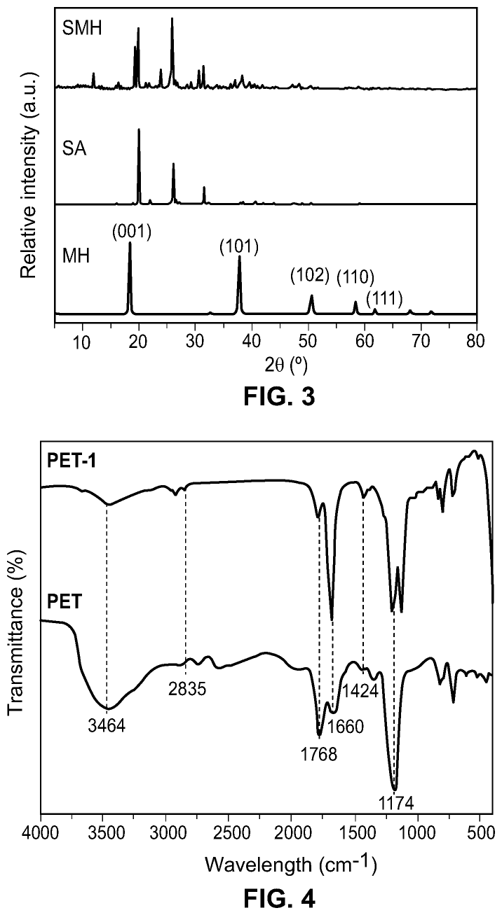

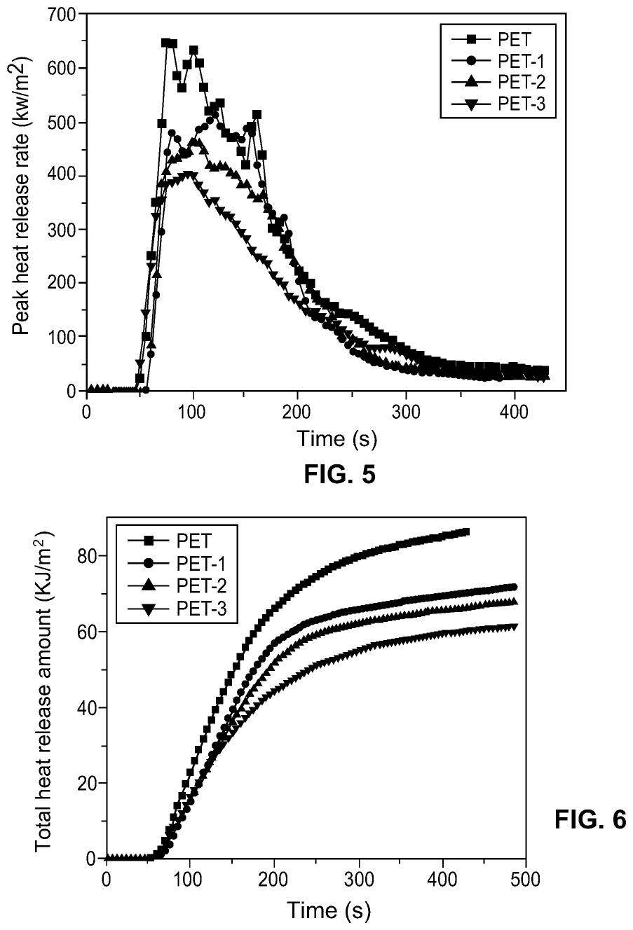

### DETAILED DESCRIPTION

A method for preparing a PET composite material containing a functionalized organic magnesium salt is further described below through specific embodiments.

### Embodiment 1

(1) Organic functionalization of magnesium hydroxide: 16.28 g of succinic acid was taken and dissolved in 300 mL of distilled water, 4 g of magnesium hydroxide was added after the succinic acid was completely dissolved, magnetic stirring was carried out for 2 h at 90° C., distilled water was removed, and vacuum drying was performed to obtain a white powdery solid which was a functionalized organic magnesium salt SMH.

(2) Preparation of a flame-retardant PET composite material: 830.65 g of terephthalic acid, 403.44 g of ethylene glycol, 0.29 g of antimony trioxide and 24.6 g of SMH were taken, mixed and added into a 1.5 L reaction kettle. The air tightness of the instrument was checked, nitrogen was slowly introduced to eliminate air in the kettle, and the temperature was controlled at 200° C. for esterification reaction; when the column top temperature was less than 100° C., the esterification reaction was finished, and water was drained. Then the system entered the polycondensation stage. After half vacuum polycondensation for 0.5 h, the temperature was slowly raised to 260° C. for high vacuum polymerization. When the stirring power no longer increased, the reaction was finished, a material was discharged and subjected to grain-sized dicing, and the master batch was dried at 130° C. for 13 h to remove moisture, so that a flame-retardant PET composite material was obtained.

(3) Properties of the flame-retardant PET composite material: flame retardancy: an oxygen index of 25% and a peak heat release rate of 600 kW/m². Mechanical properties: bending strength of 70 MPa and bending modulus of 1600 MPa.

### Embodiment 2

(1) Organic functionalization of magnesium oxide: 34 g of p-aminobenzoic acid was dissolved in 500 mL of distilled water; 5 g of magnesium oxide was added after the p-aminobenzoic acid was completely dissolved, magnetic stirring was carried out for 3 h at 80° C., distilled water was removed, and vacuum drying was performed to obtain a white powdery solid which was a functionalized organic magnesium salt PMO.

(2) Preparation of a flame-retardant PET composite material: 830.65 g of terephthalic acid, 403.44 g of ethylene glycol, 0.29 g of antimony trioxide and 50 g of PMO were taken, mixed and added into a 1.5 L reaction kettle. The air tightness of the instrument was checked, nitrogen was slowly introduced to eliminate air in the kettle, and the temperature was controlled at 220° C. for esterification reaction; when the column top temperature was less than 100° C., the esterification reaction was finished, and water was drained. Then the system entered the polycondensation stage. After half vacuum polycondensation for 0.5 h, the temperature was slowly raised to 275° C. for high vacuum polymerization. When the stirring power no longer increased, the reaction was finished, and a material was discharged and subjected to grain-sized dicing. The master batch was dried at 140° C. for 14 h to remove moisture, so that a flame-retardant PET composite material was obtained.

(3) Properties of the flame-retardant PET composite material: flame retardancy: an oxygen index of 27% and a peak heat release rate of 500 kW/m². Mechanical properties: bending strength of 75 MPa and bending modulus of 1700 MPa.

### Embodiment 3

(1) Organic functionalization of magnesium carbonate: 11.25 g of pimelic acid was taken and dissolved in 500 mL of distilled water, 3 g of magnesium carbonate was added after the pimelic acid was completely dissolved, magnetic stirring was carried out for 5 h at 90° C., distilled water was removed, and vacuum drying was performed to obtain a white powdery solid which was an organic magnesium salt PMC.

(2) Preparation of a flame-retardant PET composite material: 830.65 g of terephthalic acid, 403.44 g of ethylene glycol, 0.29 g of antimony trioxide and 74 g of PMC were taken, mixed and added into a 1.5 L reaction kettle. The air tightness of the instrument was checked, nitrogen was slowly introduced to eliminate air in the kettle, and the temperature was controlled at 240° C. for esterification reaction; when the column top temperature was less than 100° C., the esterification reaction was finished, and water was drained. Then the system entered the polycondensation stage. After half vacuum polycondensation for 1.0 h, the temperature was slowly raised to 280° C. for high vacuum polymerization. When the stirring power no longer increased, the reaction was finished, and a material was discharged and subjected to grain-sized dicing. The master batch was dried at 160° C. for 15 h to remove moisture, so that a flame-retardant PET composite material was obtained.

(3) Properties of the flame-retardant PET composite material: flame retardancy: an oxygen index of 28% and a peak heat release rate of 450 kW/m². Mechanical properties: bending strength of 75 MPa and bending modulus of 1760 MPa.

### Embodiment 4

(1) Organic functionalization of magnesium borate: 6.5 g of phosphorous acid was taken and dissolved in 200 mL of distilled water, 6 g of magnesium borate was added after the phosphorous acid was completely dissolved, magnetic stirring was carried out for 4 h at 90° C., distilled water was removed, and vacuum drying was performed to obtain a white powdery solid which was an organic magnesium salt PMB.

(2) Preparation of a flame-retardant PET composite material: 830.65 g of terephthalic acid, 403.44 g of ethylene glycol, 0.29 g of antimony trioxide and 98 g of PMB were taken, mixed and added into a 1.5 L reaction kettle. The air tightness of the instrument was checked, nitrogen was slowly introduced to eliminate air in the kettle, and the temperature was raised to be controlled at 240° C. for esterification reaction; when the column top temperature was less than 100° C., the esterification reaction was finished, and water was drained. Then the system entered the polycondensation stage. After half vacuum polycondensation for 1.0 h, the temperature was slowly raised to 275° C. for high vacuum polymerization. When the stirring power no longer increased, the reaction was finished, and a material was discharged and subjected to grain-sized dicing. The master batch was dried at 160° C. for 16 h to remove moisture, so that a flame-retardant PET composite material was obtained.

(3) Properties of the flame-retardant PET composite material: flame retardancy: an oxygen index of 27% and a peak heat release rate of 400 kW/m². Mechanical properties: bending strength of 60 MPa and bending modulus of 1500 MPa.

### COMPARATIVE EXAMPLE

### Preparation of a PET Composite Material

(1) 830.65 g of terephthalic acid, 403.44 g of ethylene glycol and 0.29 g of antimony trioxide were mixed and added into a 1.5 L reaction kettle. The air tightness of the instrument was checked, nitrogen was slowly introduced to eliminate air in the kettle, and the temperature was controlled at 230° C. for esterification reaction; when the column top temperature was less than 100° C., the esterification reaction was finished, and water was drained. Then the system entered the polycondensation stage. After half vacuum polycondensation for 0.5 h, the temperature was slowly raised and controlled at 275° C. for high vacuum polymerization. When the stirring power no longer increased, the reaction was finished, and a material was discharged and subjected to grain-sized dicing. The master batch was dried at 120° C. for 12 h to remove moisture.

(2) Properties of the flame-retardant PET composite material: flame retardancy: an oxygen index of 24% and a peak heat release rate of 650 kW/m². Mechanical properties: bending strength of 65 MPa and bending modulus of 1560 MPa.

1. A method for preparing a functionalized organic magnesium salt, comprising completely dissolving organic acid in distilled water to form an organic acid aqueous solution, adding an inorganic magnesium salt into the organic acid aqueous solution, magnetically stirring for 1-5 h at 70-100° C., removing distilled water, and performing vacuum drying to obtain a white powdery solid which is a functionalized organic magnesium salt.

2. The method for preparing a functionalized organic magnesium salt according to claim 1, wherein the organic acid is at least one of phosphorous acid, succinic acid, p-aminobenzoic acid and pimelic acid.

3. The method for preparing a functionalized organic magnesium salt according to claim 1, wherein the inorganic magnesium salt is magnesium hydroxide, magnesium carbonate, magnesium borate and magnesium oxide.

4. The method for preparing a functionalized organic magnesium salt according to claim 3, wherein the particle size of the inorganic magnesium salt is 400-600 nm.

5. The method for preparing a functionalized organic magnesium salt according to claim 1, wherein a molar ratio of the organic acid to inorganic particles is (0.5:1)-(3:1).

6. (canceled)

7. (canceled)

8. (canceled)

9. The method for preparing a functionalized organic magnesium salt according to claim 2, wherein a molar ratio of the organic acid to inorganic particles is (0.5:1)-(3:1).

10. The method for preparing a functionalized organic magnesium salt according to claim 3, wherein a molar ratio of the organic acid to inorganic particles is (0.5:1)-(3:1).

11. The method for preparing a functionalized organic magnesium salt according to claim 4, wherein a molar ratio of the organic acid to inorganic particles is (0.5:1)-(3:1).

12. Use of a functionalized organic magnesium salt prepared by the method according to claim 1 in preparation of a polyester composite material.

13. Use of the functionalized organic magnesium salt according to claim 1 in preparation of a polyester composite material, wherein terephthalic acid, ethylene glycol, antimony trioxide and the functionalized organic magnesium salt are added into a reaction kettle, and esterification reaction is carried out at 210-240° C. and 0.2-0.35 MPa under nitrogen atmosphere; after the esterification is finished, the system enters a vacuum polycondensation stage: the temperature is first controlled at 260-280° C. for low vacuum polycondensation for 0.5-1.5 h, and then the temperature is raised to 270-280° C. for high vacuum polycondensation; when the stirring power no longer continues to increase, the reaction is finished, a material is discharged and subjected to grain-sized dicing, and the master batch is dried at 120-160° C. for 12-24 h to remove moisture, so that a polyester composite material is obtained.

14. Use of the functionalized organic magnesium salt according to claim 1 in preparation of a flame-retardant polyester composite material, wherein a molar ratio of the terephthalic acid to the ethylene glycol is (1:1.2)-(1:1.5).

15. Use of the functionalized organic magnesium salt according to claim 1 in preparation of a flame-retardant polyester composite material, wherein the consumption of the functionalized organic magnesium salt is 1%-10% of the total mass of the polyester.

16. Use of the functionalized organic magnesium salt according to claim 1 in preparation of a flame-retardant polyester composite material, wherein the consumption of the catalyst antimony trioxide is 1/10000-2/10000 of the molar weight of the terephthalic acid.
